(19) United States
(12) Patent Application Publication
(10) Pub. No.: US 2014/0277364 A1
(43) Pub. Date: Sep. 18, 2014

(54) SYSTEMS AND METHODS FOR COMPRESSING, CRIMPING AND LOADING A STENT

(71) Applicants: PERITEC BIOSCIENCES LTD., Cleveland, OH (US); THE CLEVELAND CLINIC FOUNDATION, Cleveland, OH (US)

(72) Inventors: Timur P. Sarac, Orange Village, OH (US); Christian S. Eversull, Palo Alto, CA (US); Stephen Leeflang, Sunnyvale, CA (US)

(73) Assignees: PERITEC BIOSCIENCES LTD., Cleveland, OH (US); THE CLEVELAND CLINIC FOUNDATION, Cleveland, OH (US)

(21) Appl. No.: 14/201,814

(22) Filed: Mar. 8, 2014

Related U.S. Application Data
(60) Provisional application No. 61/779,244, filed on Mar. 13, 2013.

(51) Int. Cl. A61F 2/95

(52) U.S. Cl. CPC A61F 2/95
USPC 623/1.12; 29/452

(57) ABSTRACT

A stent compression and crimping system includes a sheath having a proximal end, a distal end comprising a removable funnel, and a lumen extending between the proximal and distal ends. The system further includes a guidewire extending through the sheath's lumen. The system also includes a plurality of elongate wires. Each wire has a proximal end attached to a collar, a distal free end, and a wire body extending between the proximal end and the distal end. The plurality of elongate wires is collectively disposed about the guidewire in an operative configuration.

### RELATED APPLICATION

This application claims priority from U.S. Provisional Application No. 61/779,244, filed 13 Mar. 2013, the subject matter of which is incorporated herein by reference in its entirety.

### TECHNICAL FIELD

The present invention relates to systems and methods for compressing, crimping and loading a medical device into a delivery apparatus.

### BACKGROUND

Stents are generally implantable cylindrical shaped devices that are radially expandable to hold open a segment of a blood vessel or other anatomical lumen. A stent acts as a scaffold to support the lumen in an open position. Various configurations of stents include coils and cylindrical tubes defined by a mesh or other interconnected segments. Balloon-expandable stents are mounted on a collapsed balloon and have a diameter smaller than when the stents are deployed. Stents can also be self-expanding, assuming a final diameter when deployed from a delivery device.

In order to load a stent into a tube-like delivery apparatus and ultimately deliver the stent into the patient, the stent is generally first collapsed to reduce its diameter or annular perimeter. One method of accomplishing this is using a funnel attached to the tube-like delivery apparatus to cause a gradual reduction in the diameter or annular perimeter of the stent. Optimally, the funnel should uniformly collapse the stent to the smallest diameter desired (e.g., small enough to fit into a delivery tube of a delivery apparatus) without damaging the stent.

### SUMMARY

According to an embodiment of the present invention, a stent compression and crimping system is provided. A sheath has a proximal end, a distal end comprising a removable funnel, and a lumen extending between the proximal and distal ends. The system further comprises a guidewire extending through the sheath's lumen. The system also includes a plurality of elongate wires. Each wire has a proximal end attached to a collar, a distal free end, and a wire body extending between the proximal end and the distal end. The plurality of elongate wires is collectively disposed about the guidewire in an operative configuration.

According to an embodiment of the present invention, a method of compressing and crimping a stent is provided. A stent compression and crimping system is provided, the system comprising a sheath, the sheath having a proximal end, a distal end comprising a removable funnel, and a lumen extending between the proximal and distal ends. A guidewire extends through the lumen of the sheath. A plurality of elongate wires are provided, with each wire having a proximal end attached to a collar, a distal free end, and a wire body extending between the proximal end and the distal end. The sheath is clamped to a clamp at a position proximal to the funnel. A stent is placed onto the guidewire. The guidewire extends through the lumen of the sheath and through the funnel. The plurality of wires is disposed about the guidewire and stent. The plurality of wires is positioned against the stent. The proximal end of the stent is placed into the funnel. The guidewire is pulled to allow the plurality of wires to draw the stent into the funnel and through the clamp, the clamp crimping the stent and the funnel compressing the stent.

### BRIEF DESCRIPTION OF THE DRAWINGS

FIG. 1 is a perspective view of an embodiment of the present invention;

FIG. 2 is a partial perspective view of the embodiment of FIG. 1;

FIGS. 3-9 depict an example sequence of loading operation of the embodiment of

FIG. 1; and

FIGS. 10-12 depict an example sequence of deployment operation of the embodiment of FIG. 1.

### DETAILED DESCRIPTION

The present invention provides systems and methods for compressing and crimping a medical device and loading the device onto a delivery apparatus. Although the medical device can be any suitable medical device that desirably has a reduction in its diameter prior to insertion or implantation in a patient, the medical device will be described herein as being a stent. More specifically, the medical device is described herein as being a self-expanding stent.

The disclosure herein refers to the term “substantially” with respect to certain geometric shapes and configurations. By “substantially” is meant that the shape or configuration of the described component, feature or element need not have the mathematically exact described shape or configuration, but can have a shape that is recognizable by one skilled in the art as generally or approximately having the described shape or configuration. Also, the disclosure herein refers to an “operative configuration.” This is the configuration of the system when the medical device has been inserted into the patient and is being steered to the target site. Further, as used herein with respect to a described component, the terms “a,” “an,” and “the” include at least one or more of the described component unless otherwise indicated. In addition, it will be understood that when an element is referred to as being “on,” “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with, or “directly contacting” another element, there are no intervening elements present. It will also be appreciated by those of skill in the art that references to a structure or feature that is disposed “adjacent” another feature may have portions that overlap or underlie the adjacent feature.

Referring to FIG. 1, an embodiment of the present invention includes a stent compression and crimping system 10 comprising a sheath 12 having a proximal end (not shown), a distal end 14 comprising a removable funnel 16, and a lumen extending between the proximal and distal ends. Sheath 12 can be fabricated from any suitable biocompatible material such as a polymeric material. For example, the sheath 12 may be at least partially fabricated from polytetrafluoroethylene (PTFE). System 10 further comprises a guidewire 18 extending through the sheath's lumen and configured for longitudinal motion through the lumen. The distal tip of guidewire 18 is depicted in the figures as including a nosecone 19 but could have other configurations.

System 10 also includes a plurality of elongate wires 20 collectively connected to a collar at the wires' proximal end. In particular, each wire 20 is a strand having a proximal end attached to a collar 22 (seen in FIG. 4), a distal free end 24, and a wire body 26 extending between the proximal end and the distal end. The term “wire” is used herein to indicate a thin, elongated thread or rod of material, which may have any desired degree of rigidity as appropriate for a particular use application of the present invention, and which is significantly larger along a longitudinal axis thereof than in a direction lateral to the longitudinal axis.

As seen in FIGS. 1 and 2, the plurality of elongate wires 20 is collectively disposed about guidewire 18 and a stent 40 as shown in FIG. 4 in an operative configuration and as described in more detail below.

Although FIGS. 1 and 2 depict four wires 20, a system 10 can include any number of wires. Further, as can be seen in FIG. 1, a system can include four wires 20a, 20b, 20c, and 20d (having corresponding free ends 24a, 24b, 24c, 24d and wire bodies 26a, 26b, 26c, 26d), each of any length, cross-section, configuration, and/or any other properties, relative to the remaining wires and coordinating with or independently from the properties of any other wires. For example, to help with space allocation inside the sheath 12, every other wire 20 (relative to the circumference of the sheath) may be of a first length, in alternating arrangement with wires 20 of the second length. However, the wires could have other relative lengths as well, or could all be the same length, as desired for a particular use environment of the present invention. In an example embodiment, the plurality of wires circumferentially surrounds guidewire 18 and stent 40 (as described below) in an operative configuration. Further, the wires 20 may be spaced substantially equidistantly apart around a circumference of the guidewire. For example, in embodiments with four wires, the wires may be spaced 90 degrees apart.

One or more of the wires 20 may have an inner, longitudinal, elongate edge facing the guidewire and stent that is relatively tapered. The term “tapered” is used herein to indicate a thin, keen edge that has a significantly smaller lateral width than another portion of the wire located laterally further from the guidewire.

Each wire 20 may have a substantially arcuate lateral cross-sectional shape, as shown in FIG. 1 and, especially, the magnified portion of FIG. 2—for example, the plurality of wires may collectively have a lateral cross-sectional shape that is round/circular and configured to collectively surround the guidewire 18.

The wires 20 can be fabricated from any suitable material that allows the wires to draw a stent into the funnel of an exemplary system as described in more detail below. For example, the wire 20 could be at least partially fabricated from a biocompatible metal or polymeric material, such as stainless steel or nitinol, for example.

Referring to FIG. 3, according to an example method of compressing and crimping a stent 40 using the system 10 described herein, sheath 12 is first clamped to a stationary surface, for example, such as via a table clamp 41, at a location proximal to funnel 16. Table clamp 41 acts to crimp stent 40 as the system is drawn proximally. Table clamp 41 defines an aperture 51 through which sheath 12 is inserted.

A stent is positioned over guidewire 18 as shown in FIG. 4 and at least the bodies 26 of the plurality of wires 20 surround guidewire 18 and stent 40.

Referring to FIG. 5, at least the proximal portion of stent 40 is then loaded into funnel 16. In particular, FIG. 5 illustrates the plurality of wires 20 being urged against the outer surface of stent 40 by funnel 16. The plurality of wires 20 helps draw stent 40 into funnel 16 as the user pulls the plurality of wires, along with the stent 40 contained therein, toward the right, in the orientation of FIG. 5. Lateral pressure of the plurality of wires 40 against the stent 40 starts the process of laterally compressing the stent. As such, and as described above, one or more of the wires 20 may have a tapered/arcuate inside edge facing stent 40 to facilitate capture and guidance of stent 40 into funnel 16.

As seen in FIG. 6, guidewire 18 and stent 40, along with wires 20, are pulled proximally (toward the right, in the orientation of FIG. 6) through aperture 51 of clamp 41 to allow clamp 41 to crimp, or laterally compress, stent 40. The funnel 16 assists the wires 20 to gradually compress stent 40, as the stent passes through the funnel and into the reduced-diameter (reduced from the funnel mouth) sheath 12. In addition to helping to urge stent 40 longitudinally into funnel 16, wires 20 also provide structural support to the stent 40 within the sheath 12, and can at least partially act as a scaffold to assist with crimping (lateral compression) of the stent 40, as the stent 40 and surrounding wires 20 pass through the funnel 16 and the aperture 51 of table clamp 41. The wires 20 may act in a “guiding rail” type manner to help prevent grinding or damage to the stent 40 during passage of the stent through the funnel 16 and the aperture 51 of table clamp 41.

As shown in FIGS. 7 and 8, after stent 40 assumes a compressed and crimped configuration in sheath 12, funnel 16 may be removed. In FIG. 7, funnel 16 is removed by cutting sheath 12 with a pair of scissors at a location proximal to funnel 16. (Care should be exercised to avoid cutting guidewire 18 or other structures of the system during removal of the funnel 16.) Funnel 16 could be removed in other ways, as desired. For example, funnel 16 could be snap-fitted onto the distal end of sheath 12 and could be simply tugged off. Alternatively, sheath 12 could have a frangible section proximal to funnel 16 to facilitate selective detachment of the funnel from the sheath. Once funnel 16 has been removed (in any suitable manner, whether or not described here) as seen in FIG. 8, nosecone 19 of guidewire 18 may be retracted into sheath 12 as seen in FIG. 9. Optionally, though not shown, the wires 20 and/or any associated structures could be removed from this “finished” loaded/crimped stent 40, for example, by being pulled toward the right (in the orientation of FIG. 9) and out from the sheath 12.

FIGS. 10 through 12 show an example method of deploying compressed and crimped stent 40 in a blood vessel 82. FIG. 10 illustrates retracting sheath 12 and wires 20 to allow stent 40 to start to assume its expanded configuration. FIG. 11 illustrates further retracting sheath 12 and wires 20 to further release the stent 40 to self-expand in the blood vessel 82. FIG. 12 illustrates even further retracting sheath 12, guidewire 18, and the plurality of wires 20 to fully expose the self-expanded stent 40 in blood vessel 82. Once the apparatus has reached the fully deployed configuration of FIG. 12, the sheath 12, guidewire 18, and the plurality of wires 20 can be removed from the blood vessel 82.

While aspects of the present invention have been particularly shown and described with reference to the preferred embodiment above, it will be understood by those of ordinary skill in the art that various additional embodiments may be contemplated without departing from the spirit and scope of the present invention. For example, the specific methods described above for using the described system 10 are merely illustrative; one of ordinary skill in the art could readily determine any number or type of components, sequences of steps, or other means/options for operating a system in a manner substantially similar to those shown and described herein. Any of the described structures and components could be integrally formed as a single piece or made up of separate sub-components, with either of these formations involving any suitable stock or bespoke components and/or any suitable material or combinations of materials. Though certain components described herein are shown as having specific geometric shapes, all structures of the present invention may have any suitable shapes, sizes, configurations, relative relationships, cross-sectional areas, or any other physical characteristics as desirable for a particular application of the present invention. Any structures or features described with reference to one embodiment or configuration of the present invention could be provided, singly or in combination with other structures or features, to any other embodiment or configuration, as it would be impractical to describe each of the embodiments and configurations discussed herein as having all of the options discussed with respect to all of the other embodiments and configurations. The system 10 could be at least partially disposable or intended for one-time use, possibly by including a sacrifice feature (not shown) rendering the instrument unusable after an initial use—this may be particularly helpful in a medical use environment if the apparatus is not intended for repeat sterilization and reuse. A device or method incorporating any of these features should be understood to fall under the scope of the present invention as determined based upon the claims below and any equivalents thereof.

Other aspects, objects, and advantages of the present invention can be obtained from a study of the drawings, the disclosure, and the appended claims.

What is claimed is:

1. A stent compression and crimping system comprising:
a sheath having a proximal end, a distal end comprising a removable funnel, and a lumen extending between the proximal and distal ends;
a guidewire extending through the lumen of the sheath; and
a plurality of elongate wires, each wire having a proximal end attached to a collar, a distal free end, and a wire body extending between the proximal end and the distal end, the plurality of elongate wires collectively disposed about the guidewire in an operative configuration.

2. The stent compression and crimping system of claim 1, wherein at least one of the free end and wire body of the plurality of wires has a tapered edge facing the guidewire.

3. The stent compression and crimping system of claim 1, wherein the plurality of elongate wires is four wires.

4. The stent compression and crimping system of claim 1, wherein the plurality of elongate wires each has an arcuate lateral cross-sectional shape.

5. The stent compression and crimping system of claim 1, wherein the sheath comprises PTFE.

6. The stent compression and crimping system of claim 1, wherein the sheath has a frangible section proximal to the funnel providing selective detachment of the funnel from the sheath.

7. The stent compression and crimping system of claim 1, wherein the guidewire has a distal tip comprising a nosecone.

8. The stent compression and crimping system of claim 1, further comprising a catheter coupled to the proximal end of the sheath, the catheter having a lumen extending longitudinally therethrough.

9. A method of compressing and crimping a stent comprising:
providing a stent compression and crimping system comprising:
a sheath having a proximal end, a distal end comprising a removable funnel, and a lumen extending between the proximal and distal ends;
a guidewire extending through the lumen of the sheath;
a plurality of elongate wires, each wire having a proximal end attached to a collar, a distal free end, and a wire body extending between the proximal end and the distal end;
clamping the sheath to a clamp at a position proximal to the funnel;
placing a stent onto the guidewire, the guidewire extending through the lumen of the sheath and through the funnel, the plurality of wires disposed about the guidewire and stent;
positioning the plurality of wires against the stent;
placing the proximal end of the stent into the funnel; and
pulling the guidewire to allow the plurality of wires to draw the stent into the funnel and through the clamp, the clamp crimping the stent and the funnel compressing the stent.

10. The method of claim 10, wherein clamping the sheath comprises clamping the sheath to a stationary structure.

11. A method of delivering a stent comprising the method of compressing and crimping a stent according to claim 10 and further comprising:
removing the funnel;
drawing the guidewire into sheath; and
retracting the sheath to expose the stent.

12. The method of claim 11, wherein the guidewire has a distal tip comprising a nosecone and drawing the guidewire into the sheath comprises retracting the nosecone proximally into the sheath.
